(19) United States
(12) Patent Application Publication
(10) Pub. No.: US 2018/0071307 A1
(43) Pub. Date: Mar. 15, 2018

(54) MEDICINAL COMPOSITION FOR ALLEVIATING ENDOMETRIOSIS AND COMPLICATIONS THEREOF AND USE OF THE SAME

(71) Applicant: NATIONAL SUN YAT-SEN UNIVERSITY, Kaohsiung (TW)

(72) Inventors: Jim Jinn-Chyuan SHEU, KAOHSIUNG (TW); Yin-Yi CHANG, KAOHSIUNG (TW); Chih-Mei CHEN, KAOHSIUNG (TW); An-Jen CHIANG, KAOHSIUNG (TW)

(21) Appl. No.: 15/705,235

(22) Filed: Sep. 14, 2017

Related U.S. Application Data
(60) Provisional application No. 62/394,219, filed on Sep. 14, 2016.

(51) Int. Cl. A61K 31/551; A61K 31/501; A61K 9/00

(52) U.S. Cl. CPC A61K 31/551; A61K 9/0019; A61K 31/501

(57) ABSTRACT

The present invention relates to a medicinal composition for alleviating endometriosis and complications thereof and a use of the same. The medicinal composition includes an inhibitor of ribosome biosynthesis and a pharmaceutically acceptable carrier. When the medicinal composition is administrated to a cell of endometrium or surrounding tissue thereof, endometriosis and its complications can be alleviated.

### RELATED APPLICATIONS

This application claims priority to U.S. Provisional Application Ser. No. 62/394,219, filed Sep. 14, 2016, which is herein incorporated by reference.

### BACKGROUND

### Field of Invention

The present invention relates to a medicinal composition. More specifically, the present invention relates to a medicinal composition for alleviating endometriosis and complications thereof and use of the same through inhibiting ribosome biosynthesis.

### Description of Related Art

Endometriosis is a benign, yet debilitating, gynecological disease associated with chronic pelvic pain, dysmenorrhea and infertility. Affecting about 10% of reproductive-age females, endometriosis causes abnormal growth of endometrium-like tissues outside the uterine cavity. These benign peritoneal surface growths, which can invade ectopically, mimic the progression of metastasis in malignant cancer, which is accompanied by angiogenesis and cell migration. Histopathological observations and genetic analyses have shown that both endometrioid and clear cell ovarian carcinomas arise from endometriosis. Although several hypotheses have been proposed regarding the etiology of endometriosis, the exact pathogenesis of the disease remains unclear. Multiple factors may be involved in an individual's susceptibility to endometriosis, including hormone aberrations, abnormal immune responses, environmental factors and individual anatomy, as well as genetic or epigenetic predisposition.

Small nucleolar RNAs (snoRNAs) are non-coding RNAs with longer mature sequences (60-300 nt) than microRNAs (miRNAs). They can be divided into two major classes with distinct signature sequences, box C/D or box H/ACA, functioning as guiding components for small ribonucleoprotein particles, catalyzing rRNA 2′-O-methylation and pseudouridylation, respectively, through complementary recognition sequences. In the eukaryotic cell nucleolus, ribosomal RNA is post-transcriptionally edited by snoRNAs and subsequently cleaved to yield 18S, 5.8S and 28S rRNAs. These fragments are assembled into the mature large and small RPs, preceding translocation to the cytoplasm. Both snoRNAs and RPs are key regulators in ribosome biogenesis, which is especially crucial for cell cycle progression. Recent studies suggested that upregulation of snoRNAs and RPs controls human tumor development. Perturbation of blocking upstream signals, ribosome assembly by RNA polymerase I inhibition or snoRNA/RP silencing can arrest cell proliferation and induce apoptosis, and has been suggested as a novel strategy against malignant diseases.

However, there is no effective strategy to alleviating endometriosis and its complications. Accordingly, there is an urgent need to develop a novel composition to alleviate endometriosis and its complications.

### SUMMARY

The invention provides a medicinal composition for alleviating endometriosis and complications thereof, which comprises an inhibitor of ribosome biosynthesis, thereby for alleviating endometriosis and its complications.

Moreover, the invention also provides an inhibitor of ribosome biosynthesis in use of preparation of a medicinal composition for alleviating endometriosis and complications thereof.

According to the aforementioned aspect, the invention provides a medicinal composition for alleviating endometriosis and complications thereof. In an embodiment, the medicinal composition includes an inhibitor of ribosome biosynthesis and a pharmaceutically acceptable carrier, in which the inhibitor of ribosome biosynthesis can includes at least one of an RNA polymerase I inhibitor; an inhibitor of an rDNA gene upstream regulator; and an inhibitor of an rRNA processing regulator.

In the aforementioned embodiment, the RNA polymerase I inhibitor can include a quinolone compound.

In the aforementioned embodiment, the inhibitor of the rDNA gene upstream regulator can include mTOR/c-Myc inhibitor.

In the aforementioned embodiment, the inhibitor of the rRNA processing regulator can include SNORD inhibitor and RP inhibitor involved in ribosome biosynthesis and/or inhibitors against the upstream signaling, for example, small nucleolar RNA C/D box 116 (SNORD116) inhibitor, ribosomal large P protein 2 (RPLP2) inhibitor, ribosomal protein L26 (RPL26) inhibitor, RPL38 inhibitor, ribosomal protein S25 (RPS25) inhibitor, RPS27 inhibitor, RPS28 inhibitor and any combination thereof.

According to the another aspect, the invention provides an inhibitor of ribosome biosynthesis in use of preparation of a medicinal composition for alleviating endometriosis and complications thereof. In an embodiment, the inhibitor of ribosome biosynthesis can includes at least one of an RNA polymerase I inhibitor; an inhibitor of an rDNA gene upstream regulator; and an inhibitor of an rRNA processing regulator. In the aforementioned embodiment, the medicinal composition can be subjected to a cell having a genotype CC at a SNP accession number of rs11614913.

In the aforementioned embodiment, the RNA polymerase I inhibitor can include a quinolone compound.

In the aforementioned embodiment, the inhibitor of the rDNA gene upstream regulator can include mTOR/c-Myc inhibitor.

In the aforementioned embodiment, the inhibitor of the rRNA processing regulator can include SNORD inhibitor and RP inhibitor and/or inhibitors against the upstream signaling. In an example, the SNORD inhibitor and RP inhibitor can include but be not limited to small nucleolar RNA C/D box 116 (SNORD116) inhibitor, ribosomal protein large P2 (RPLP2) inhibitor, ribosomal protein L26 (RPL26) inhibitor, RPL38 inhibitor, ribosomal protein S25 (RPS25) inhibitor, RPS27 inhibitor, RPS28 inhibitor and any combination thereof.

In the aforementioned embodiment, the complications of endometriosis can include ovarian cancer, endometrial cancer and/or cervical cancer.

With application to the medicinal composition for alleviating endometriosis, which includes an inhibitor of ribosome biosynthesis and a pharmaceutically acceptable carrier, thereby alleviating endometriosis and its complications.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The disclosure can be more fully understood by reading the following detailed description of the embodiment, with reference made to the accompanying drawings as follows.

FIG. 1 is depicted to microarray data from the GEO databank (GSE6364) that was used to assess expression of selected snoRNA and RP genes in endometriosis lesions (E) and normal endometrium (N).

FIGS. 2A to 2B illustrate ribosome biogenesis upregulation during endometriosis progression.

FIG. 3 illustrates a diagram showing cellular factors and inhibitors involved in ribosome biosynthesis.

FIGS. 4A to 4E illustrate that mTOR/c-Myc inhibition suppresses cell growth and mobility of ovarian clear cells according to an embodiment of the present invention.

FIGS. 5A to 5D illustrate that RNA polymerase 1 inhibition suppresses cell growth and mobility of ovarian clear cells.

FIG. 6 illustrates a scheme showing the timeline of procedures performed on donor and recipient mice.

FIG. 7 illustrates a bar diagram showing lesion numbers among the mice treated with the drug (CX: CX-5461; GSK: GSK2126458) or vehicle only [V(CX): vehicle for CX-5461; V(GSK): vehicle for GSK)] according to an embodiment of the present invention.

FIGS. 8A and 8B illustrates bar diagrams showing inflammatory cells (small peritoneal macrophages, FIG. 8A; neutrophils, FIG. 8B) in abdominal cavity of mice having endometriosis with different treatments according to an embodiment of the present invention.

FIGS. 9A to 9C illustrate behavior analyses of pain relief of mice having endometriosis with different treatments according to an embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As aforementioned, the present invention provides a medicinal composition for alleviating endometriosis and complications thereof and a use of the same. When the medicinal composition including an inhibitor of ribosome biosynthesis is applied to cells having a specific genotype at a specific SNP site, endometriosis and its complications can be alleviated.

Typically, the “medicinal composition” as discussed hereinafter can include an inhibitor of ribosome biosynthesis and a pharmaceutically acceptable carrier. In an embodiment, the inhibitor of ribosome biosynthesis can includes at least one of an RNA polymerase I inhibitor; an inhibitor of an rDNA gene upstream regulator; and an inhibitor of an rRNA processing regulator.

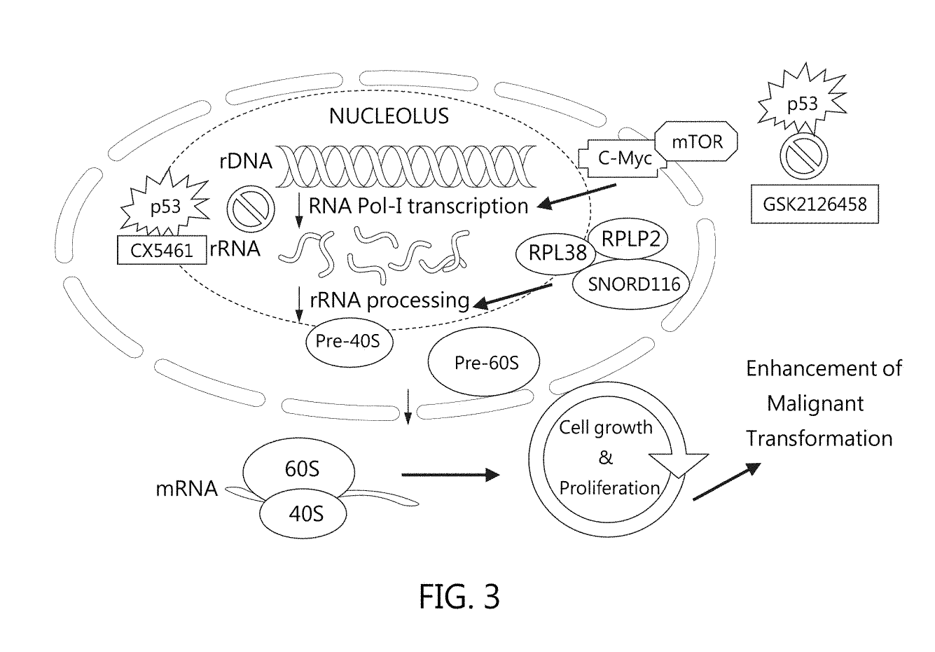

Please refer to FIG. 3, which illustrates a brief overview showing cellular factors and inhibitors involved in ribosome biosynthesis. The biogenesis of the ribosome machinery is a highly coordinated process, which is composed of the synthesis and import of ribosomal proteins into the nucleus, synthesis and processing of ribosomal RNA (rRNA), assembly of ribosomal proteins and subsequent transport of the mature subunits into the cytoplasm. Most of these events take place in the nucleolus, except for 5S rRNA synthesis (which occurs in the nucleoplasm, not shown in FIG. 3) and synthesis of ribosomal proteins (which occurs in the cytoplasm). An inhibitor of an rDNA gene upstream regulator (for example, GSK2126458), an RNA polymerase I inhibitor (for example, CX5461) and an inhibitor of an rRNA processing regulator can interfere or suppress factors involved in rDNA gene upstream regulator (for examples, mTOR/c-Myc), the transcription (for example, RNA polymerase I) and the rRNA processing regulator. In FIG. 3, rDNA, ribosomal DNA; RPL26, ribosomal protein L26; RPLP2, ribosomal protein large P2; RPS25, ribosomal protein S25; and SNORD116, small nucleolar RNA C/D box 116.

In an example, the rRNA processing regulator can be SNORD genes and RP genes involved in ribosome biosynthesis, for example, SNORD116, RPLP2, RPL26, RPL38, RPS25, RPS27, RPS28 and so on. It should be noted that, the aforementioned SNORD genes and RP genes are merely illustrative and are not intended to limit the present invention. It should be noted that, the aforementioned genes are merely illustrative and are not intended to limit the present invention.

In an example, the RNA polymerase I inhibitor can include a quinolone compound, examples of which can include but not limited to CX-5461, oxolinic acid and its sails, nalidixic acid and its sails, coumermycin A1, novobiocin and other functionally-similar compounds.

In the aforementioned example, the inhibitor of the rDNA gene upstream regulator can include mTOR/c-Myc inhibitor. Examples of mTOR inhibitor can include, for example, rapamycin and its derivatives, mTORC1/mTORC2 dual inhibitor (TORCdls) and so on. Examples of rapamycin and its derivatives can include but not limited to omipalisib (or called as 2,4-difluoro-N-[2-methoxy-5-[4-(4-pyridazinyl)-6-quinolinyl]-3-pyridinyl]benzenesulfonamide; GSK2126458), rapamycin (or called as sirolimus), deforolimus [(or called as 42-(dimethylphosphinate) rapamycin; AP23573)], everolimus (RAD001) and temsirolimus (CCI-779). Examples of mTORC1/mTORC2 dual inhibitor (TORCdls) can include but not limited to sapanisertib [INK128, or called as 3-(2-amino-5-benzoxazolyl)-1-(1-methylethyl)-1H-pyrazolo[3,4-D]pyrimidin-4-amine], AZD8055, AZD2014 and so on.

In the aforementioned example, examples of the inhibitor of the rDNA gene processing regulator can include SNORD inhibitor and RP inhibitor involved in ribosome biosynthesis and/or inhibitors against the upstream signaling, for example, small nucleolar RNA C/D box 116 (SNORD116) inhibitor, ribosomal protein large P2 (RPLP2) inhibitor, ribosomal protein L26 (RPL26) inhibitor, RPL38 inhibitor, ribosomal protein S25 (RPS25) inhibitor, RPS27 inhibitor, RPS28 inhibitor and any combination thereof. In the clinical specimens of endometriosis and its complications, the expressions of SNORD 116, RPLP2, RPS27, RPS25, RPL26, RPL38 and RPS28 are usually upregulated, enhancing ribosome biosynthesis, thereby causing endometriosis and its complications. It should be noted that, the aforementioned inhibitors to specific genes are merely illustrative and are not intended to limit the present invention.

The “pharmaceutically acceptable carrier” as discussed hereinafter refers to an inactive ingredient itself, which can be a carrier, diluent, adjuvant and/or mediator for delivering the active ingredient to a subject; an additive added into the composition for improving the processing or storing properties of the composition; an excipient or other substance for allowing or facilitating the administration at a suitable dose conveniently. The aforementioned pharmaceutically acceptable carrier should not destroy the pharmaceutical activity of the active ingredient, and it is nontoxic when delivering enough therapeutic dose of the active ingredient.

The suitable “pharmaceutically acceptable carrier”, which can be ones well known by one skilled in the manufacturation of the medicinal composition, includes but is not limited to a buffer, diluent, disintegrant, binder, adhesive, humectant, polymer, lubricant, glidant; an additive for masking or neutralizing the unpleasant taste or odor; a dye, fragrance and additive for improving the appearance of the composition. Specific examples of the pharmaceutically acceptable carrier can include but be not limited to citrate buffer, phosphate buffer, acetate buffer, bicarbonate buffer, stearic acid, magnesium stearate, magnesium oxide, sodium and calcium salts of phosphoric and sulfuric acid, magnesium carbonate, talc, gelatin, arabic gum, sodium alginate, pectin, dextrin, mannitol, sorbitol, lactose, sucrose, starch, gelatin, cellulose material (such as cellulose esters of alkanoic acids and cellulose alkyl esters), low melting point wax, cocoa butter, amino acids, urea, alcohols, ascorbic acid, phospholipids, proteins (such as serum albumin), ethylenediaminetetraacetic acid (EDTA), dimethyl sulfoxide (DMSO), sodium chloride or other salts, liposome, glycerol or powder, polymers (such as polyvinyl pyrrolidone, polyvinyl alcohol, and polyethylene glycol) and other pharmaceutically acceptable substances.

In application, the inhibitor of ribosome biosynthesis and the medicinal composition including the same can be administrated via a subcutaneous injection, an in situ injection, an intravenous injection or an oral administration to a subject, specifically reducing or inhibiting the activity of ribosome biosynthesis, thereby alleviating endometriosis and its complications. More specifically, as evidenced by the in vitro cell test, when the inhibitor of ribosome biosynthesis and the medicinal composition including the same are applied on cancer cells for a desired duration such as 24 to 96 hours, the activities of the cancer cells, for example, DNA synthesis, specific gene expression or cell cycle of cancer, can be inhibited, even resulting in apoptosis of cancer cells.

One of uses of the medicinal composition can be subjected to a cell, thereby alleviating endometriosis and its complications.

The “complications of endometriosis” as discussed hereinafter can include but be not limited to ovarian cancer, endometrial cancer and/or cervical cancer. The “alleviating endometriosis and complications thereof” as discussed hereinafter can be achieved by inhibiting the ribosome biosynthesis, followed by suppressing the cell proliferation and migration, arresting the cell cycle and leading to apoptosis, so as to alleviate endometriosis and complications thereof.

The “upregulated” or “upregulation” as discussed hereinafter denotes to increase the amount of particular cellular components such as DNA, RNA protein and the like. On the contrary, the “downregulated” or “downregulation” as discussed hereinafter denotes to decrease the amount of particular cellular components such as DNA, RNA protein and the like.

Thereinafter, various applications of the medicinal composition for alleviating endometriosis and complications thereof and the use of the same will be described in more details referring to several exemplary embodiments below, while not intended to be limiting. Thus, one skilled in the art can easily ascertain the essential characteristics of the present invention and, without departing from the spirit and scope thereof, can make various changes and modifications of the invention to adapt it to various usages and conditions.

### EXAMPLE

### 1. Cell Culture, Cell Sorting and Functional Study

Endometrial cells [HECIA (BCRC 60552 or ATCC HTB-112) and RL95-2 (BCRC 60103 or ATCC CRL-1671)] and ovarian clear cells [ES-2 (BCRC 60067 or ATCC CRL-1978) and TOV-21G (BCRC 60407 or ATCC CRL-11730)] were purchased from Bioresource Collection and Research center (BCRC), Taiwan, or American Type Culture Collection (ATCC). BCRC is located in Food Industry Research and Development Institute (FIRDI), Hsin-Chu, Taiwan. ATCC is located in 10801 University Blvd, Manassas, Va. 20110, U.S.A.

For anti-ribosome biogenesis assays, including cell growth, cell migration and cell cycle analysis, ovarian clear cells were maintained for five days in culture medium with or without RNA polymerase I inhibitor, CX-5461 (Selleckchem, Houston, Tex.) or GSK2126458 (GlaxoSmithKline, Middlesex, United Kingdom).

### 2. Immunofluorescence Staining

Eight paraffin blocks showing continuous histopathological transition from distant endometriosis, contiguous atypical endometriosis and ovarian clear cell carcinomas were selected for sectioning. Immunofluorescence staining was performed to detect active cell nucleoli and ribosome biogenesis activity using 1:100 rabbit anti-nucleophosmin (anti-NPM; ab52644) and anti-nucleolin (anti-NCL; ab129200) monoclonal antibodies (Abeam PLC, Cambridge, Mass.). Immunostaining was independently scored by two pathologists, and specific nucleolus staining was scored as: negative (0), weakly positive (1+), moderately positive (2+) or strongly positive (3+). This Example used a combination of the percentage of positively stained cells and the intensity of nucleolus staining for statistical analysis. The H-score=IPi xi was calculated, where i is the intensity of the stained tumor cells (0 to 3+) and Pi is the percentage of the stained tumor cells for each intensity group (0 to 100%) as disclosed in the Journal of Pathology 229(4):559-568 (2013), which is incorporated herein by reference. For discordant cases, a third investigator was brought in to score and the final intensity score was determined by the majority scores.

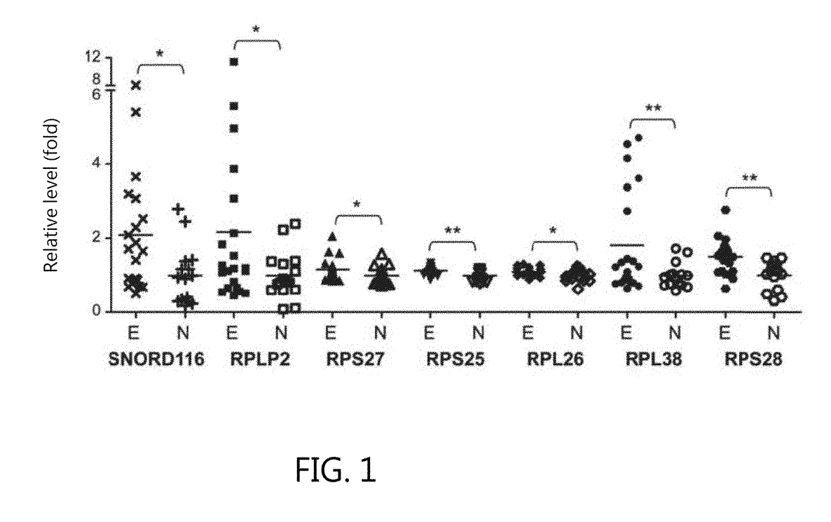

Please refer to FIG. 1, which is depicted to microarray data from the GEO databank (GSE6364) that was used to assess expression of selected snoRNA and RP genes in endometriosis lesions (n=21, represented as E) and normal endometrium (n=16, represented as N). *P<0.05; **P<0.01; ***P<0.001.

To define the clinical significance of our findings, microarray data from the GEO databank (accession number: GSE6364) were utilized to analyze expression of the selected snoRNAs and RPs in clinical endometriosis lesions and normal endometrium. Six out of eight RPs were found to be upregulated in endometriosis tissues (FIG. 1). Due to limitations of the probe-set design, small nucleolar RNA C/D box 116 (SNORD 116) was the only selected snoRNA gene found in the dataset with higher levels in endometriosis tissues (represented as E) compared to controls (represented as N). Among the selected genes, SNORD 116, RPLP2, RPL38 and 40S ribosomal protein S28 (RPS28) were most elevated ones in endometriosis patients (FIG. 1, represented as E). These results indicate an overall activation of ribosome biogenesis during endometriosis development.

### 3. Ribosome Biogenesis Upregulation Triggers Endometriosis Progression

Ribosome biogenesis is the greatest energetic and metabolic expenditure that takes place in the cell nucleolus, especially in cancer cells. Structural-functional studies have revealed that nucleolar abnormalities correlate with cancer development and represent an adaptation to the new metabolic characteristics acquired by transformed cells. Therefore, the activation of ribosome biogenesis might be a driving force triggering malignant transformation during endometriosis progression.

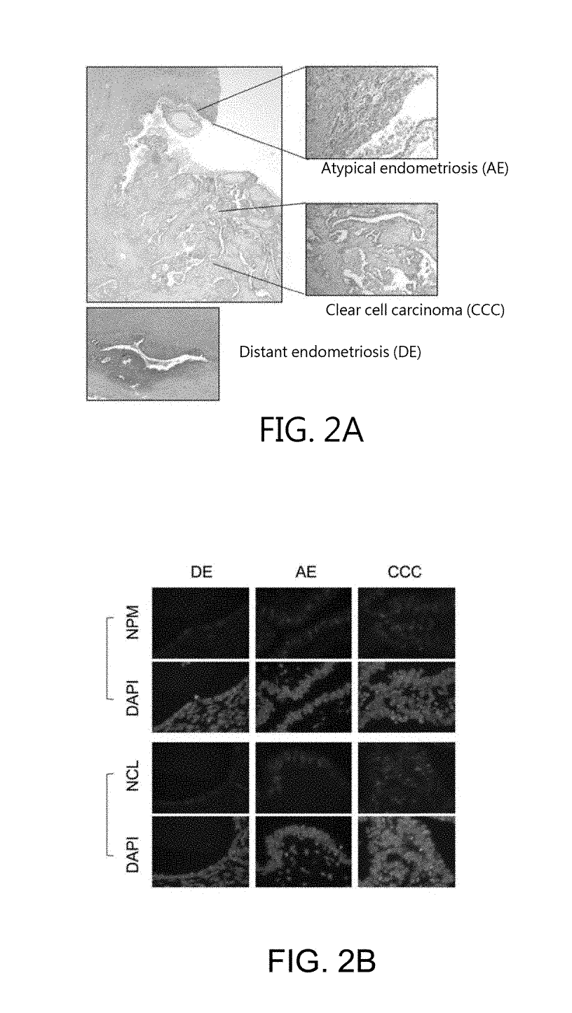

Please refer to FIGS. 2A to 2B, which illustrate ribosome biogenesis upregulation during endometriosis progression. FIG. 2A is depicted to tissue sections with contiguous atypical endometriosis and ovarian clear cell carcinoma. FIG. 2B is depicted to tissue sections prepared for anti-NPM and anti-NCL staining. Staining was scored as aforementioned. The scores of all staining images were represented as means of 100 nucleoli with standard variations. Distant endometriosis from the same patient was used as the control of FIGS. 2A to 2B. *P<0.05; **P<0.01; ***P<0.001.

To test this hypothesis, eight clear cell ovarian carcinomas were collected in this Example for immunofluorescence staining (FIG. 2A). In this Example, total active nucleoli were detected using anti-nucleophosmin (NPM) antibodies, and the dense fibrillary component (DFC), a region with highly active ribosome biogenesis, was detected using anti-nucleolin (NCL) antibodies. The results indicated that contiguous atypical endometriosis adjacent to cancer tissues had greater NCL and NPM staining intensities compared to distant endometriosis lesions (FIG. 2B). Consistent with this, cancerous tissue nucleoli had greatly enlarged total areas (anti-NPM) with activated ribosome biogenesis (anti-NCL) (FIG. 2B). Of note, the tissue blocks carrying the T/T genotype showed weaker staining intensities than those carrying the C/C genotype. The data provide evidence that increased nucleoli and enlarged DFC morphology indicate an unfavorable transformation from endometriosis to atypical endometriosis and finally ovarian cancer, endometrial cancer and/or cervical cancer.

4. rDNA Gene Upstream Inhibition Suppresses Cell Growth and Mobility of Ovarian Clear Cells

As aforementioned, upregulation of rDNA genes has been linked to accelerate cell proliferation, thus has been suggested as a therapeutic target for cancer treatment. Two ovarian clear cell carcinoma cell lines with wild type TP53 genetic status, ES-2 and TOV-2I G, were used to investigate the efficacy of anti-ribosome biogenesis therapy by treating cells with GSK2126458, an mTOR/c-Myc inhibitor.

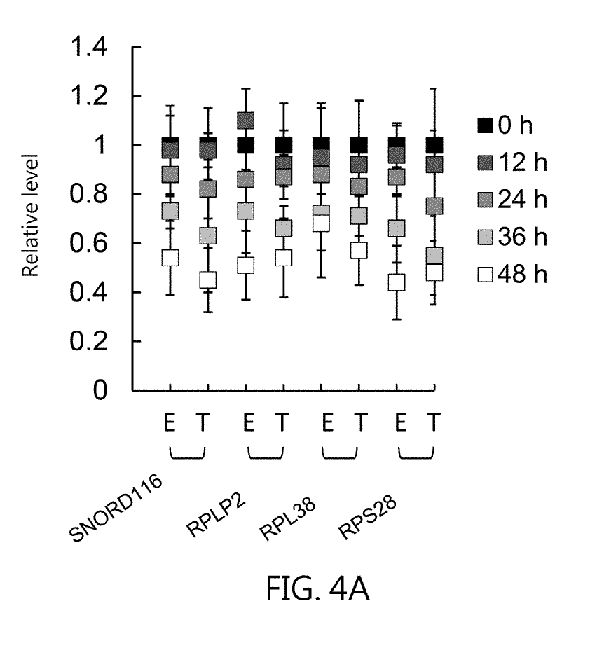

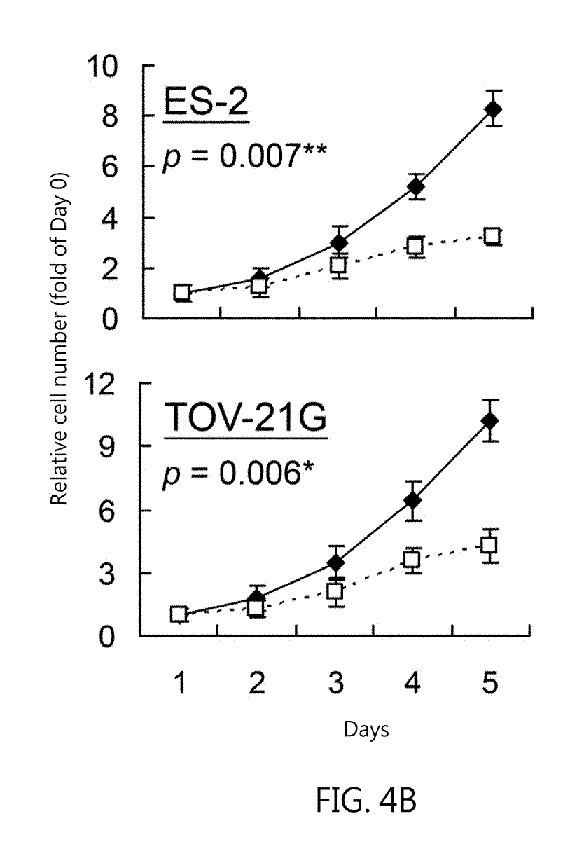

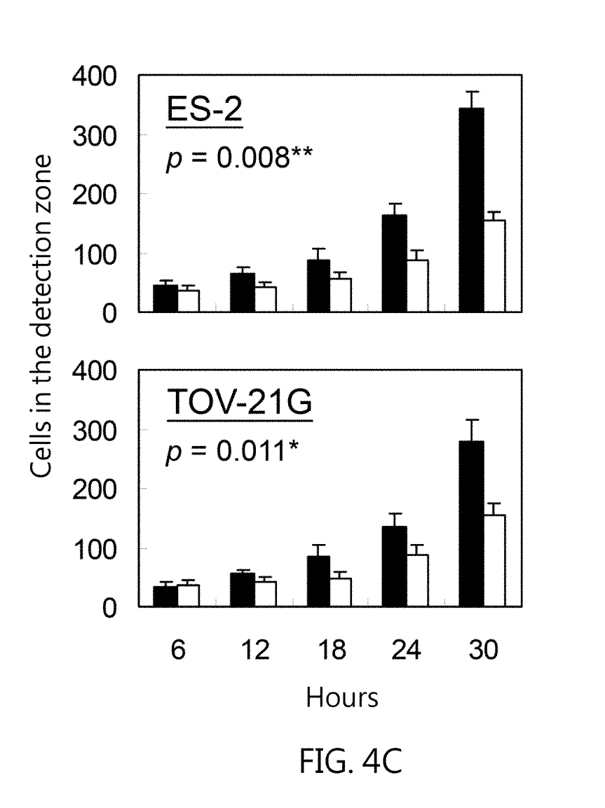

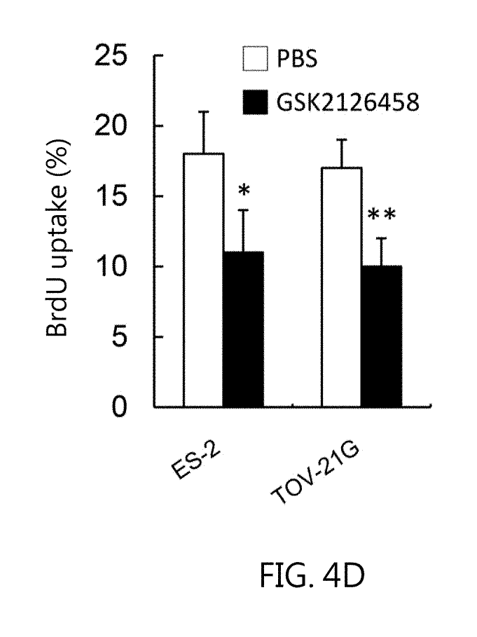

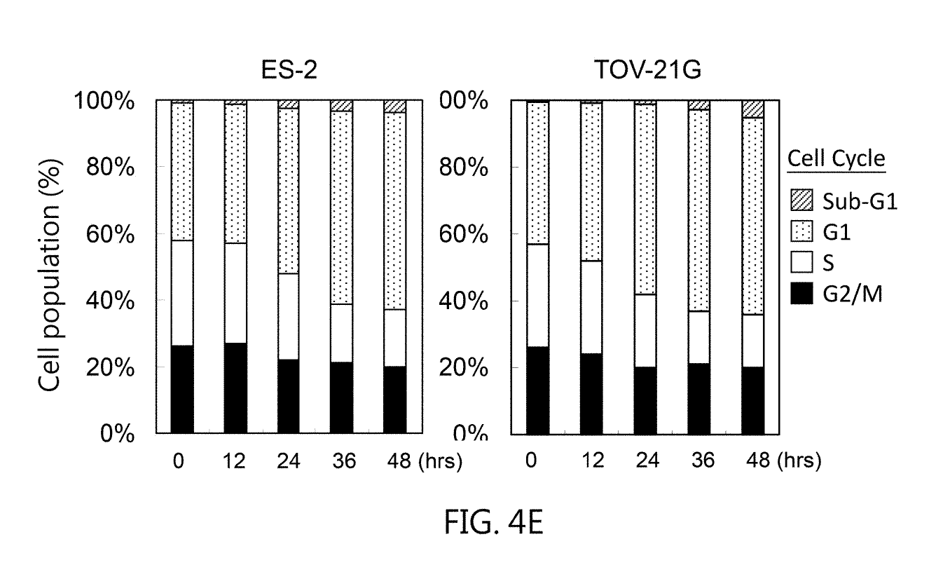

Please refer to FIGS. 4A to 4E, which illustrate that mTOR/c-Myc inhibition suppresses cell growth and mobility of ovarian clear cells according to an embodiment of the present invention. FIG. 4A is depicted to ovarian cancer cell lines, ES-2 and TOV-2IG, treated with the mTOR/c-Myc inhibitor, GSK2126458, at a final concentration of 50 nM. GSK2126458 effects on effectors of ribosome biogenesis were measured by qPCR at the indicated time points. Data were represented as means of triplicates with standard variations. FIG. 4B is depicted to GSK2126458 effects on cell growth were assessed via MTT assay. FIG. 4C is depicted to cell mobility measured using wound-healing migration assays. Cells migrating into the detection zone were counted and averaged from eight replicates. FIG. 4D is depicted to DNA synthesis (BrdU incorporation) of cells influenced by GSK2126458. FIG. 4E is depicted to cell cycle inhibited by GSK2126458 after 48 hours of treatment and analyzed by flow cytometry using propidium iodide DNA staining. The cell cycle of many cells was inhibited and arrested at G1 phase.

As aforementioned, expression of the selected snoRNAs and RPs of ovarian clear cell carcinoma, ES-2 and TOV-2IG was reduced following 24 h of CX-5461 treatment (FIG. 4A). Cell proliferation and mobility rates were decreased with inhibition of DNA synthesis after 48 h of GSK2126458 treatment (FIGS. 4B-4C). GSK2126458 treatment triggered cell arrest at G1-phase and increased cells of sub-G1 phase, implying cell death (FIG. 4E).

### 5. RNA Polymerase 1 Inhibition Suppresses Cell Growth and Mobility of Ovarian Clear Cells

As aforementioned, upregulation of snoRNAs and RPs has been linked to accelerate cell proliferation, thus has been suggested as a therapeutic target for cancer treatment. Two ovarian clear cell carcinoma cell lines with wild type TP53 genetic status, ES-2 and TOV-2I G, were used to investigate the efficacy of anti-ribosome biogenesis therapy by treating cells with CX-5461, an RNA polymerase I inhibitor.

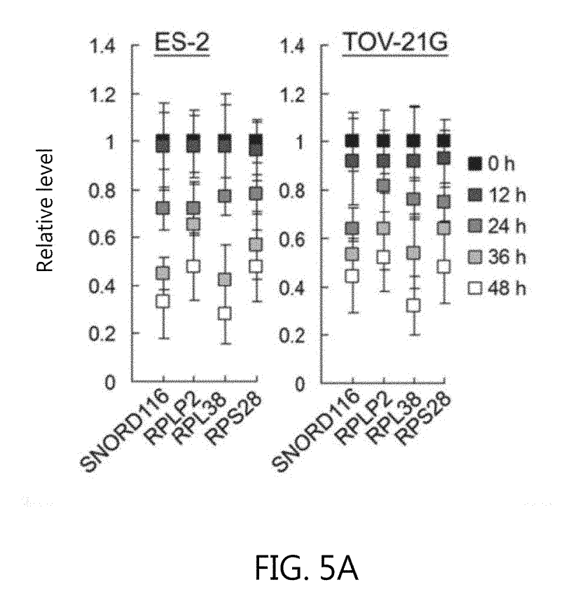

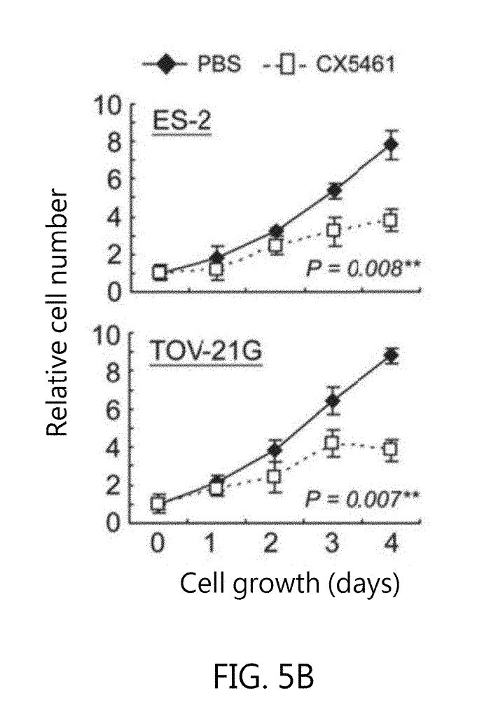

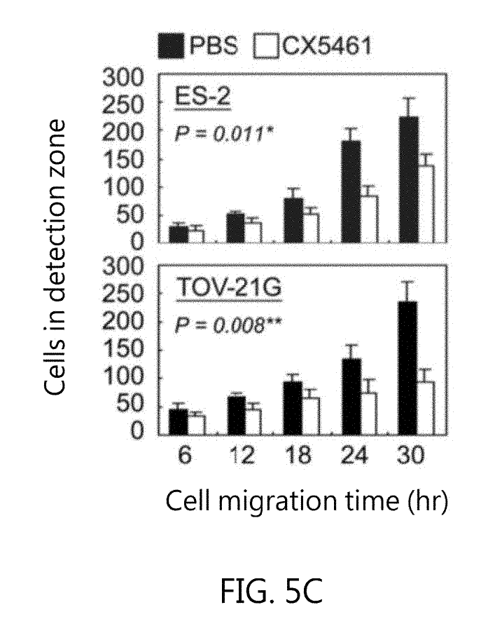

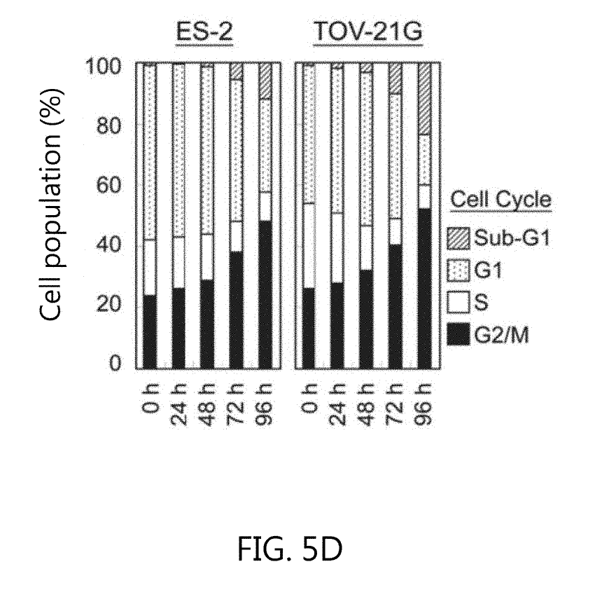

Please refer to FIGS. 5A to 5D, which illustrate that RNA polymerase 1 inhibition suppresses cell growth and mobility of ovarian clear cells. FIG. 5A is depicted to cells treated with the RNA polymerase 1 inhibitor, CX-5461, at a final concentration of 25 nM. CX-5461 effects on downstream effectors of ribosome biogenesis were measured by qPCR at the indicated time points. Data were represented as means of triplicates with standard variations. FIG. 5B is depicted to CX-5461 effects on cell growth were assessed via MTT assay. FIG. 5C is depicted to cell mobility measured using wound-healing migration assays. Cells migrating into the detection zone were counted and averaged from eight replicates. FIG. 5D is depicted to treated cells that were analyzed at the indicated time points by flow cytometry using propidium iodide DNA staining.

Expression of the selected snoRNAs and RPs reduced following 24 h of CX-5461 treatment (FIG. 5A). Cell proliferation and mobility rates were decreased with inhibition of DNA synthesis after 48 h of CX5461 treatment (FIGS. 5B-5D). CX-5461 treatment triggered cell arrest at G2/M-phase (48.1% in ES-2 cells and 52.3% in TOV-21G cells) and cell death (12.3% in ES-2 cells and 23.4% in TOV-21G cells) after 96 h of CX-5461 treatment (FIG. 5D).

6. Mouse Model of Endometriosis that Mimics Human Phenotype Confirms that Inhibition of the Ribosome Biosynthesis can Alleviate the Endometriosis and its Complications

6.1 Animals

Mature (approximately 8-week-old) female C57BL/6 mice were purchased from Harlan Laboratories (Derby, UK) and were allowed to acclimate for 1 week before surgery. Mice were maintained on standard chow and water available ad libitum and were housed in environmentally controlled facilities illuminated between 7:00 AM and 7:00 PM. All the animal procedures were performed in accordance with legal requirements.

6.2 Mouse Model of Endometriosis

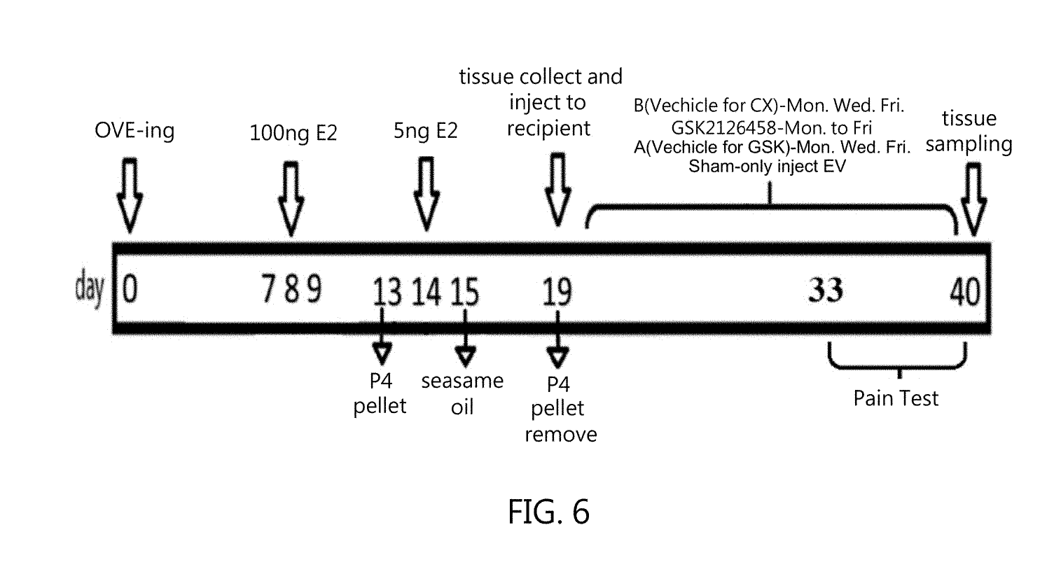

Please refer to FIG. 6, which illustrates a scheme showing the timeline of procedures performed on donor and recipient mice, as disclosed in the American Journal of Pathology 184(7):1930-1939 (2014), which is incorporated herein by reference.

Typically, menstruation was induced in adult donor mice (approximately 8 weeks of age) using a protocol developed as aforemented. In brief, long-term ovariectomized (OVE) mice (day 1) were primed with s.c. injections of 100 ng of estradiol-17β (days 7 to 9), treated with progesterone (P4; Sigma-Aldrich, Dorset, UK) delivered via a SILASTIC implant (Dow Corning Corp, Midland, Mich.) from days 13 to 19, and injected with 5 ng of estradiol-17β in sesame oil on days 13, 14, and 15. Decidualization was induced in one uterine horn using 20 mL of oil (day 15), and endometrial tissue in the process of being shed from the decidualized horn was recovered from mice culled on day 19, 4 hours after P4 withdrawal (removal of pellet) by opening the horn longitudinally in a petri dish and scraping the tissue away from the myometrial layer using a scalpel. The tissue mass was suspended in 0.2 mL of PBS and passed through a 19-gauge needle before being injected i.p. into anesthetized recipient mice (approximately 8 weeks of age) that had been previously ovariectomized and implanted with an estradiol-17β (Sigma-Aldrich) SILASTIC implant (FIG. 6). Tissue from one decidualized donor horn was used to inoculate each recipient mouse (approximately 40 mg tissue/0.2 mL PBS per mouse) on day 19. In FIG. 6, there are four groups: B, injecting recipient mice with EV once and vehicle of CX-5461 every Monday, Wednesday and Friday for three weeks; GSK, injecting recipient mice with EV once and GSK2126458 on Monday to Friday for three weeks; A, injecting recipient mice with EV once and vehicle of GSK2126458 on Monday to Friday for three weeks; Sham, injecting recipient mice with EV once (no endometriosis, hormone treatment); Naive, mice without endometriosis or treated with any hormone or drug.

Prostaglandin E2 (E2, 100 ng/100 μL or 5 ng/100 μL) was diluted in sesame oil. Estradiol valerate (EV, 500 ng/100 μL) was diluted in sesame oil. P4 pellet was fused with progesterone (P4; Sigma-Aldrich, Dorset, UK) delivered via a SILASTIC implant (Dow Corning Corp, Midland, Mich.). 0.075 mg/100 μL of GSK2126458 (GlaxoSmithKline, Middlesex, United Kingdom was dissolved in a vehicle V(GSK) composed of 1% DMSO, 30% PEG, 1% Tween-80 and 68% water] was administrated to a mice (0.025 kg/mice), five times (Monday to Friday) per week for lasting three weeks. 1.25 mg/100 μL of CX-5461 (Selleckchem, Houston, Tex.) was dissolved in a vehicle V(CX) composed of sodium phosphate monobasic (NaH₂PO₄) in water (600 mg/100 mL), pH 4.5, followed by administrating a mice (0.025 kg/mice), thrice (Monday, Wednesday and Friday) per week for lasting three weeks. Common analgesics, anesthetic and iodine were used in mice according to conventional doses during the test.

On day 33 to day 40, some behavior tests were scored from tunnel entries and pressure pain thresholds. Mice freely moved in three cages connected by tunnels, and numbers of tunnel entered by each mouse were measured in five minutes, and the average of three measures were calculated, for evaluating overall activity of each mouse. Less tunnel entries were considered as more hyperalgesia.

Pressure pain thresholds were assessed by a commercially available pain evaluation kit (Touch Test® Sensory Evaluators (North Coast Medical Inc., CA, U.S.A.). This pain evaluation kit has different probes (or needles) of different diameters, which were respectively stab the abdomen or the palmar side of the hind paw of mice. Mice were instructed to signal when the stimulus became painful, and observed which size cause painful. Threshold levels (gravity, g) were assessed before and after the test, and the average of three measures were calculated, for evaluating abdominal retraction threshold (g) or paw withdrawal threshold (g) of each mouse. Less g (gravity) value was considered as more hyperalgesia.

Three weeks after i.p. injection, recipient mice were culled (photographs of the body cavity taken and were taken the lesions carefully dissected from surrounding tissue) and tissues were either fixed in 4% normal buffered formaldehyde for histologic analysis, for counting lesion numbers in endometrium. Inflammatory cells were collected from abdominal cavity for analyzing and counting inflammatory cells (for example, small peritoneal macrophages and neutrophils).

Near the end of the (day 33 to day 40), peritoneal endometriotic lesion biopsy samples (n=24) were obtained from mice undergoing laparoscopic surgery for the treatment of endometriosis-associated pain. Biopsy samples were collected in neutral-buffered formalin for histochemical analysis.

6.3 Evaluation of Lesion Numbers

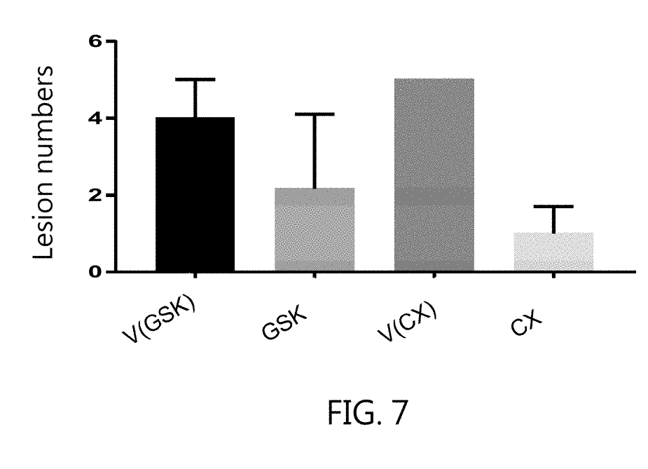

Please refer to FIG. 7, which illustrates a bar diagram showing lesion numbers among the mice treated with the drug (CX: CX-5461; GSK: GSK2126458) or vehicle only [V(CX): vehicle for CX-5461; V(GSK): vehicle for GSK)] according to an embodiment of the present invention. The lesion numbers of mice treated with drugs (CX: CX-5461; GSK: GSK2126458) were significantly reduced compared to the ones treated with the vehicles [V(CX): vehicle for CX-5461; V(GSK): vehicle for GSK)], respectively. Therefore, the drugs, GSK2126458 and CX-5461, for inhibit upstream of ribosome biosynthesis and RNA polymerase I was applied to mice having endometriosis, endometriosis lesions could be reduced.

6.4 Evaluation of Inflammatory Cells in Abdominal Cavity

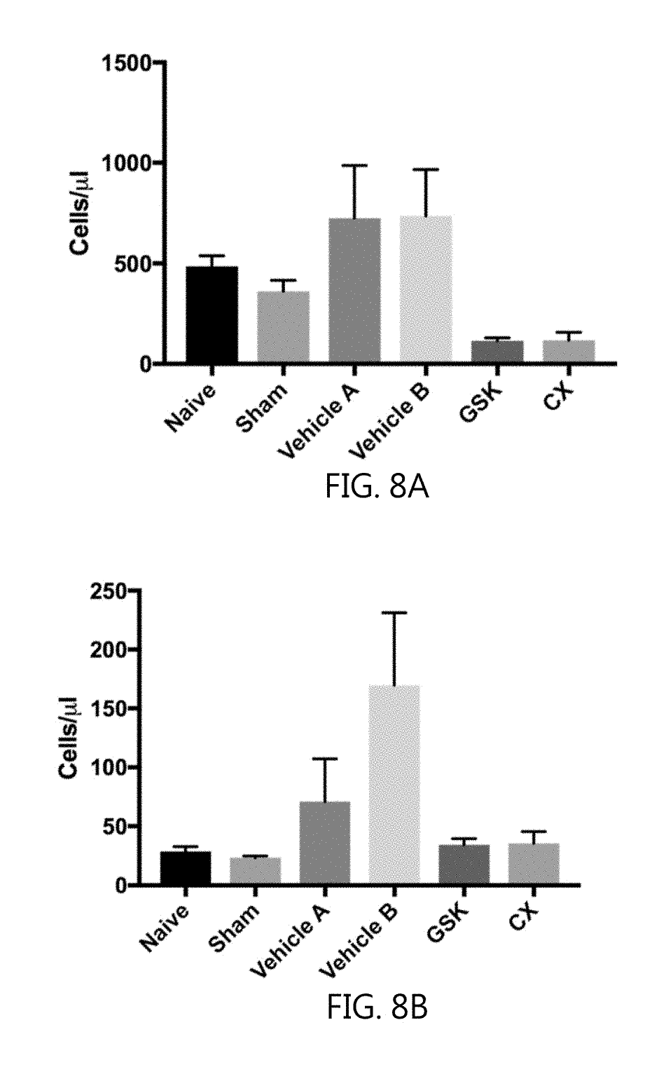

Please refer to FIGS. 8A and 8B, which illustrates bar diagrams showing inflammatory cells (small peritoneal macrophages, FIG. 8A; neutrophils, FIG. 8B) in abdominal cavity of mice having endometriosis treated with drugs (CX: CX-5461; GSK: GSK2126458), vehicles (Vehicle A: vehicle for GSK; Vehicle B: vehicle for CX-5461), sham (no endometriosis, hormone treatment) or naive (without endometriosis or treated with any hormone or drug) according to an embodiment of the present invention. Comparing with groups of naive, sham and vehicles, inflammatory cells (small peritoneal macrophages, FIG. 8A; neutrophils, FIG. 8B) in the abdominal cavity of mice treated with drugs (CX: CX-5461; GSK: GSK2126458) were significantly reduced. Therefore, the GSK2126458 and CX-5461, for inhibit upstream of ribosome biosynthesis and RNA polymerase I was applied to mice having endometriosis, inflammation could be reduced.

6.5 Evaluation of Behaviors of Pain Relief

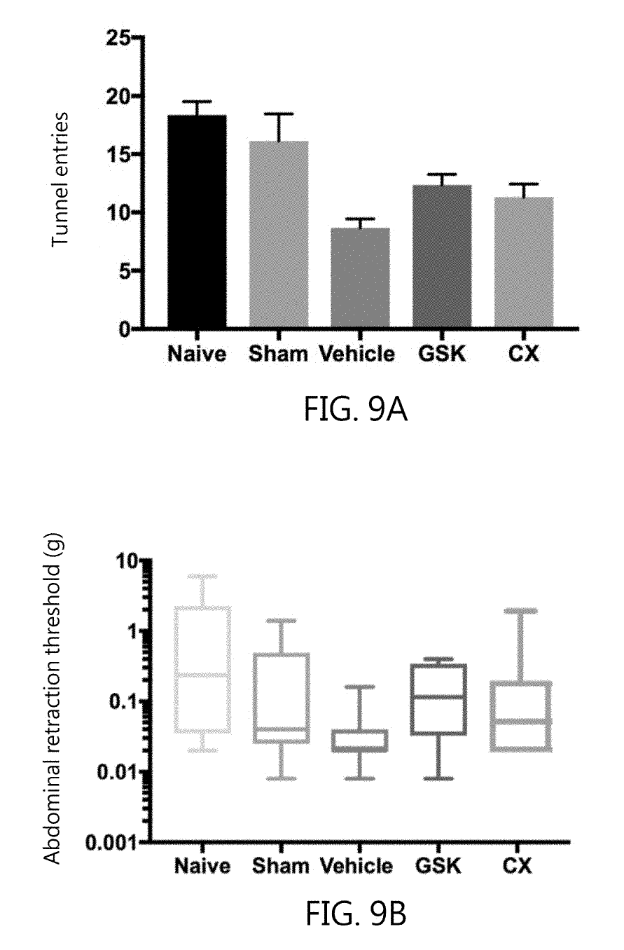

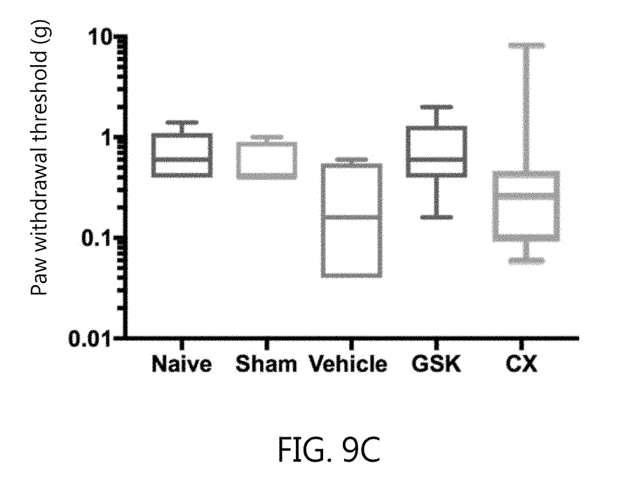

Please refer to FIGS. 9A to 9C, which illustrate behavior analyses of pain relief of mice having endometriosis with different treatments according to an embodiment of the present invention.

FIG. 9A illustrates an overall activity by measuring the tunnel entries of each mouse in five minutes. The mice treated with drugs (CX: CX-5461; GSK: GSK2126458) or without treatments (naive and sham) were more active than the ones treated with vehicles (average of vehicle for GSK and vehicle for CX), implying that the endometriosis-related pain was relieved. Therefore, the GSK2126458 and CX-5461, for inhibit upstream of ribosome biosynthesis and RNA polymerase I was applied to mice having endometriosis, the endometriosis-related pain was relieved and the overall activity could be recovered.

FIGS. 9B and 9C illustrate mechanical hyperalgesia of abdomen (FIG. 9B) and hind paw (FIG. 9C) of mice with different probes of different diameters according to an embodiment of the present invention. The mice treated with drugs (CX: CX-5461; GSK: GSK2126458) or without treatments (naive and sham) had higher abdominal retraction threshold (FIG. 9B) and paw withdrawal threshold (FIG. 9C), implying that mice treated with drugs relieved pain and were more tolerant to pain than the ones treated with vehicles (average of vehicle for GSK and vehicle for CX). Therefore, the GSK2126458 and CX-5461, for inhibit upstream of ribosome biosynthesis and RNA polymerase I was applied to mice having endometriosis, the endometriosis-related pain was relieved.

These snoRNAs and RPs were generally upregulated in endometriosis lesions as compared to normal endometrium, suggesting that active ribosome biogenesis in cell nucleoli drives endometriosis. Immunofluorescent staining against NPM and NCL further confirmed that changes in nucleolar integrity correlate with aggressive progression from endometriosis to atypical endometriosis and clear cell ovarian cancer. Treatment with inhibitors of ribosome biosynthesis (CX-5461; GSK: GSK2126458), inhibited cell proliferation and migration in ovarian clear cells, and triggered cell cycle arrest and apoptosis.

On the other hand, an overall increase in snoRNAs and RPs as the effecting genes of ribosome biogenesis suggests enhanced ribosome activity crucial for cell proliferation and the expansion of endometriosis tissue. These data indicated that enhanced expression of SNORD 116, RPLP2, RPS27, RPS25, RPL26, RPL38 and RPS28, all of which were upregulated in clinical specimens. Florescent staining against both NPM (active nucleoli) and NCL (DFC regions in the nucleoli) revealed that ribosome biogenesis was more active in contiguous atypical endometriosis than in distant endometriosis, and greater staining patterns can be found in cancerous tissues. Thus, the present application suggests the point that active ribosome biogenesis could be a driving force for malignant transformation during endometriosis development and progression.

Previous studies support this application that overexpression of RPs contributes to cell transformation and could be utilized as prognostic markers for human cancers. Ribosomal P protein (RPLP0, RPLP1, RPLP2) expression was previously shown to correlate with invasiveness and metastasis in gynecologic tumors. Although limited information is available regarding the functions of SNORD116, a C/D box snoRNA that controls the 2′-O-ribose methylation of rRNAs, accumulating evidence implicates snoRNAs in the control of cell fate and carcinogenesis through a bypassing of ribosomal/oncogenic stress responses. For example, upregulation of C/D box snoRNAs was reported as a common feature in breast and prostate cancers.

In addition to their key functions in ribosome assembly and protein synthesis, snoRNAs and RPs play novel roles outside cell nucleoli, regulating the activity and function of other oncogenes or tumor suppressors. Several effectors of ribosome biogenesis, including RPS27, RPL26, RPS25 and RPL26, participate in the MDM2-p53 feedback loop upon ribosomal/oncogenic stress. Disruption of rRNA synthesis and editing/processing, such as by chemical inhibition of RNA polymerase I, triggers MDM2 degradation and stabilizes/activates p53, leading to cell apoptosis or senescence. Similarly, specific siRNAs against C/D box snoRNAs suppressed cell cycle progress and reduced tumor growth by activating p53. With emerging roles for RNA processing in cancer development, targeting rDNA transcription and the nucleolus is a feasible cancer treatment strategy, and has shown efficacy against hematological malignancies.

Notably, human cancers exhibit differential sensitivity to anti-RNA polymerase 1 therapy, depending largely on TP53 status. Genetic analysis has shown that TP53 mutations rarely occur (˜10%) in endometriosis-associated ovarian cancers, and are considered as late genetic events during endometriosis progression if they occur. The present application indicates enhanced ribosome biogenesis activity during endometriosis development, and this activity is more pronounced during the malignant transition. This suggests that anti-RNA polymerase I therapy may be efficacious for treating endometriosis and associated ovarian cancers.

In summary, it is necessarily supplemented that, specific inhibitors of ribosome biosynthesis, expression of specific genes, specific criteria for classification of clinical disease severity, specific analysis models or specific evaluating methods are exemplified for clarifying the medicinal composition for alleviating endometriosis and complications thereof and the use of the same. However, as is understood by a person skilled in the art, other inhibitors of ribosome biosynthesis, expression of other genes, other criteria for classification of clinical disease severity, other analysis models or other evaluating methods can be also adopted in the medicinal composition for alleviating endometriosis and complications thereof and the use of the same of the present invention. For examples, the medicinal composition of the present invention can beneficially alleviate endometriosis, atypical endometriosis and ovarian cancer, so as to alleviate the complications related to endometriosis, such as endometrial cancer and/or cervical cancer.

According to the embodiments of the present invention, the medicinal composition for alleviating endometriosis and complications thereof of the present invention advantageously includes an inhibitor of ribosome biosynthesis and a pharmaceutically acceptable carrier. When the medicinal composition is applied to cells, endometriosis and its complications can be alleviated. Therefore, the inhibitor of ribosome biosynthesis can be used for preparation of a medicinal composition for alleviating endometriosis and complications thereof.

Although the present invention has been described in considerable detail with reference to certain embodiments thereof, other embodiments are possible. Therefore, the spirit and scope of the appended claims should not be limited to the description of the embodiments contained herein.

What is claimed is:

1. A medicinal composition for alleviating endometriosis and complications thereof, comprising an inhibitor of ribosome biosynthesis and a pharmaceutically acceptable carrier, wherein the inhibitor of ribosome biosynthesis is at least one selected from the group consisting of an RNA polymerase I inhibitor; an inhibitor of an rDNA gene upstream regulator; and an inhibitor of an rRNA processing regulator, thereby being administrated to a cell of endometrium or surrounding tissue thereof.

2. The medicinal composition of claim 1, wherein the RNA polymerase I inhibitor comprises a quinolone compound.

3. The medicinal composition of claim 1, wherein the inhibitor of the rDNA gene upstream regulator comprises mTOR/c-Myc inhibitor.

4. The medicinal composition of claim 1, wherein the inhibitor of the rDNA gene processing regulator comprises SNORD genes and RP genes.

5. The medicinal composition of claim 1, wherein the SNORD genes and the RP genes comprise small nucleolar RNA C/D box 116 (SNORD116) inhibitor, ribosomal P protein 2 (RPLP2) inhibitor, RPL26 inhibitor, RPL38 inhibitor, ribosomal protein S25 (RPS25) inhibitor, RPS27 inhibitor, RPS28 inhibitor and/or any combination thereof, respectively.

6. An inhibitor of ribosome biosynthesis in use of preparation of a medicinal composition for alleviating endometriosis and complications thereof, wherein the inhibitor of ribosome biosynthesis is at least one selected from the group consisting of an RNA polymerase I inhibitor; an inhibitor of an rDNA gene upstream regulator; and an inhibitor of an rRNA processing regulator.

7. The inhibitor of ribosome biosynthesis in use of preparation of the medicinal composition for alleviating endometriosis and complications thereof of claim 6, wherein the RNA polymerase I inhibitor comprises a quinolone compound.

8. The inhibitor of ribosome biosynthesis in use of preparation of the medicinal composition for alleviating endometriosis and complications thereof of claim 6, wherein the inhibitor of the rDNA gene upstream regulator comprises mTOR/c-Myc inhibitor.

9. The inhibitor of ribosome biosynthesis in use of preparation of the medicinal composition for alleviating endometriosis and complications thereof of claim 6, wherein the inhibitor of the rDNA gene processing regulator comprises SNORD inhibitor and RP inhibitor.

10. The inhibitor of ribosome biosynthesis in use of preparation of the medicinal composition for alleviating endometriosis and complications thereof of claim 9, wherein the SNORD inhibitor and RP inhibitor comprise SNORD116 inhibitor, RPLP2 inhibitor, RPL26 inhibitor, RPL38 inhibitor, RPS25 inhibitor, RPS27 inhibitor, RPS28 inhibitor and/or any combination thereof, respectively.

11. The inhibitor of ribosome biosynthesis in use of preparation of the medicinal composition for alleviating endometriosis and complications thereof of claim 6, wherein the complications of the endometriosis comprises ovarian cancer, endometrial cancer and/or cervical cancer.
